(19) United States
(12) Patent Application Publication
(10) Pub. No.: US 2008/0234206 A1
(43) Pub. Date: Sep. 25, 2008

(54) Prodrugs of Phentermine

(75) Inventor: Travis Mickle, Coralville, IA (US)

Correspondence Address: DARBY & DARBY P.C., P.O. BOX 770, Church Street Station, New York, NY, 10008-0770, US

(73) Assignee: SHIRE LLC, Florence, KY (US)

(21) Appl. No.: 12/065,639

(22) PCT Filed: Sep. 13, 2006
(86) PCT No.: PCT/US06/35354

§ 371 Date: Apr. 21, 2008

Related U.S. Application Data
(60) Provisional application No. 60/716,057, filed on Sep. 13, 2005.

(51) Int. Cl. A61K 38/05; A61K 31/137; A61K 38/06; A61P 3/04

(52) U.S. Cl. 514/19; 514/655; 514/18

(57) ABSTRACT

The invention relates to compositions of amino acid and peptide conjugates comprising phentermine. Phentermine is covalently attached to at least one amino acid via its amine group to the N-terminus, the C-terminus, a side chain of the peptide carrier. Also discussed are methods for treating obesity.

### CROSS REFERENCE RELATED APPLICATIONS

This application claims benefit under 35 U.S.C. 119(e) to U.S. Provisional application No. 60/716,057 filed Sep. 13, 2005, which is hereby incorporated by reference in its entirety.

### FIELD OF THE INVENTION

The present invention relates to pharmaceutical compounds, compositions, and methods of using the same comprising a chemical moiety attached to phentermine. These inventions provide a variety of beneficial effects including providing fast or slow release and reducing side effects associated with taking phentermine compounds and compositions. The invention also relates to methods for protecting and administering phentermine and for treating obesity and related disorders. This invention also relates to prodrugs of phentermine that improve the amount of phentermine available in the body and at the same time avoid toxic levels from being released.

### BACKGROUND OF THE INVENTION

Phentermine is an anorectic. Anorectics are used to decrease appetite by possibly changing brain levels of serotonin. Phentermine is a nervous system stimulator causing stimulation, elevation of blood pressure, and faster heart rates. Obesity, typically defined as 20% over ideal weight results or is viewed as a contributor factor to an increase in certain diseases including high cholesterol levels, heart disease, high blood pressure, gallbladder disease, type II diabetes mellitus, hardening of the arteries, and degenerative arthritis. Controlling and decreasing an individual's weight typically results in decreases in blood pressure, cholesterol levels, and an improvement in diabetes control.

Phentermine is currently available through prescription in both name brand and generic versions. Market doses include 30 mg and 37.5 mg tablets. Phentermine is generally stored in a tight container at room temperature. Phentermine is typically prescribed as a short-term drug accompanied by an diet and behavior modification/exercise routine to treat obesity. Although, some programs combine it with diet and fenfluramine (Pondimin) over longer terms in selected obesity patients.

Phentermine also has several potential side effects including diarrhea, dry mouth, constipation, an unpleasant taste, hives, impotence, palpitations, high blood pressure, fast heart rates, overstimulation, insomnia, restlessness, tremor, and dizziness. In addition, phentermine is potentially addicting. It is important that the level of phentermine remains constant to prevent adverse side effects.

One way in which to regulate the percent of phentermine in the body is by attaching amino acids and peptides to phentermine and thereby controlling the amount of phentermine released in the body. This occurs because conversion of the amino acid or peptide prodrug to its active form is limited by cleavage of the amide bond thus decreasing the potential for release of toxic levels of the active drug.

The effective delivery of phentermine is often critically dependent on the delivery system used. The importance of these systems becomes magnified when patient compliance and of phentermine stability are taken under consideration. The blunting of the phentermine “spike” through a modulated release formulation would markedly improve the safety of that drug. In general, increasing the stability of phentermine, such as prolonging shelf life or survival in the stomach, will assure dosage reproducibility and perhaps even reduce the number of dosages required which could improve patient compliance.

There remains a need for compositions that effectively deliver phentermine. There also remains a need for methods of protecting and controlling the delivery and/or release of phentermine.

Therefore, the need still exists for a drug delivery system, which enables the use of new phentermine compositions that can reduce the technical, regulatory, and financial risks associated with phentermine agents while improving their reproducibility, bioavailability, reliability, and sustained release.

The compounds of the invention may be provided in several useful forms. As such, improved methods are needed to make pharmaceutically effective phentermine compounds, compositions and methods of using the same with reduced potential for overdose and/or lower side effects.

### BRIEF DESCRIPTION OF THE DRAWINGS

It is to be understood that both the foregoing general description and the following detailed description are exemplary, but are not restrictive, of the invention.

FIG. 1 illustrates a scheme for a single amino acid conjugation with phentermine;

FIG. 2 illustrates a scheme for dipeptide conjugation with phentermine;

FIG. 3 illustrates a scheme for tripeptide conjugation with phentermine;

FIG. 4 illustrates a scheme for linker conjugation with phentermine; and

FIG. 5 illustrates a scheme for phentermine N-terminal attachment.

### DETAILED DESCRIPTION OF THE INVENTION

The invention relates to changing the pharmacokinetic and pharmacological properties of phentermine through covalent modification. Covalent attachment of a chemical moiety to phentermine may change one or more of the following: the rate of absorption, the extent of absorption, the metabolism, the distribution, and the elimination (ADME pharmacokinertic properties) of phentermine. As such, the alteration of one or more of these characteristics may be designed to provide fast or slow release. Additionally, alteration of one or more of these characteristics may reduce the side effects associated with taking phentermine.

One aspect of the invention includes phentermine conjugates that when administered at a normal therapeutic dose the bioavailability (area under the time-versus-concentration curve; AUC) of phentermine provides a pharmaceutically effective amount of phentermine. As the dose is increased, however, the bioavailability of the covalently modified phentermine relative to the parent phentermine begins to decline, particularly for oral dosage forms. At suprapharmacological doses the bioavailability of the phentermine conjugate is substantially decreased as compared to the parent phentermine. The relative decrease in bioavailability at higher doses decreases or reduces risks associated with doses of the phentermine and helps to reduce fluctuation in bioavailability.

The invention provides phentermine prodrugs comprising phentermine covalently bound to a chemical moiety. The phentermine prodrugs can also be characterized as conjugates in that they possess a covalent attachment. They may also be characterized as conditionally bioreversible derivatives (“CBDs”).

In one embodiment, the phentermine prodrug (a compound of one of the formulas described herein) may exhibit one or more of the following advantages over free phentermine. The phentermine prodrug may prevent or reduce side effects. Preferably, the phentermine prodrug provides a serum release curve that does not increase above phentermine's toxicity level when administered at higher than therapeutic doses. The phentermine prodrug may exhibit a reduced rate of phentermine absorption and/or an increased rate of clearance compared to the free phentermine. The phentermine prodrug may also exhibit a steady-state serum release curve. Preferably, the phentermine prodrug provides bioavailability but prevents Cₘₐₓ spiking, increased blood serum concentrations, or uneven release profiles associated with current controlled release phentermine products. Preferably, the prodrugs are effectively metabolized into individual amino acids by alimentary tract enzymes before reaching systemic circulation.

The invention provides covalent attachment of phentermine to a carrier peptide, also referred to as peptidic phentermine compositions. The invention covalently attaches phentermine in a peptide-linked manner, to the N-terminus, the C-terminus, or to the amino acid side chain of the carrier peptide. In a more preferred embodiment the attachment is without the use of a linker.

The carrier peptide itself may also serve as an adjuvant. In a preferred embodiment, the phentermine is covalently attached to the N-terminus or the C-terminus of the carrier peptide or amino acid, also referred to as capped phentermine compositions. In another preferred embodiment, phentermine is covalently attached directly to the amino acid side chain of the carrier peptide or amino acid; also referred to as side chain phentermine compositions.

Phentermine may be bound to one or more chemical moieties, denominated X and Z. A chemical moiety can be any moiety that decreases the pharmacological activity of phentermine while bound to the chemical moiety as compared to unbound (free) phentermine. The attached chemical moiety can be either naturally occurring or synthetic. In one embodiment, the invention provides an phentermine prodrug of Formula I:

P—Xₙ-Zₘ (I)

wherein P is an phentermine;
each X is independently a chemical moiety;
each Z is independently a chemical moiety that acts as an adjuvant and is different from at least one X;
n is an increment from 1 to 50, preferably 1 to 10; and
m is an increment from 0 to 50, preferably 0.
When m is 0, the phentermine prodrug is a compound of Formula (II):

P—Xₙ (II)

wherein each X is independently a chemical moiety.

Formula (II) can also be written to designate the chemical moiety that is physically attached to the phentermine:

P—X₁—(X)ₙ₋₁ (III)

wherein P is phentermine; X₁ is a chemical moiety, preferably a single amino acid; each X is independently a chemical moiety that is the same as or different from X₁; and n is an increment from 1 to 50.

Compounds, compositions and methods of the invention provide reduced potential for overdose and/or improve phentermine's characteristics with regard to high toxicities or suboptimal release profiles.

As used herein, the term phentermine compounds refers to a compound of formula IV and salts thereof.

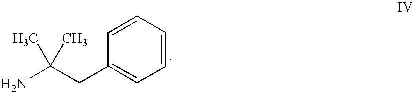

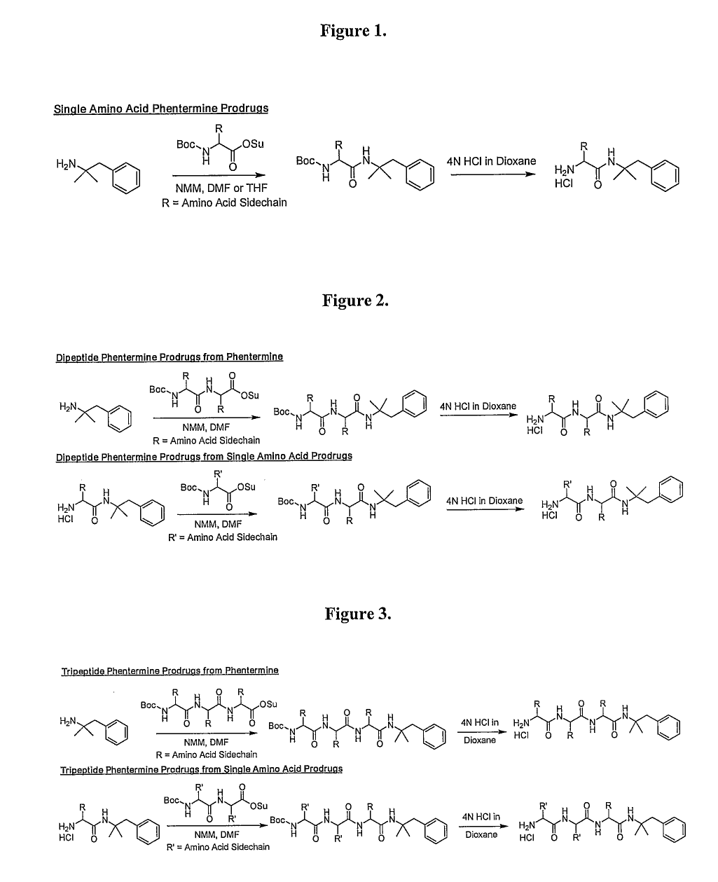

In a preferred embodiment of the invention, the location of attachment of the amine functionality of phentermine is the C-terminus of the carrier peptide as shown in FIGS. 1-3. FIG. 5 shows a phentermine compound where the N-terminal is the point of attachment.

In another embodiment of the invention, the phentermine-conjugate comprises Lys-phentermine, or X-Lys-phentermine wherein X represents a second amino acid. The second amino acid is preferably a naturally occurring amino acid. Similarly, additional preferred amino acids or peptide carriers are Ala, Gly, Leu, Val, Ile, Phe, Pro, Lys, Asp, Glu, Ser, Thr, Tyr, Ala-Ala, Gly-Gly, Phe-Phe, Glu-Glu, Tyr-Tyr, Ala-Ala-Ala, and Gly-Gly-Gly. Additionally, the first amino acid attached to phentermine is preferably Ala, Gly, Lys, Asp, Glu, Ser and Glu-Glu.

In another embodiment of the invention, for modulated delivery or increased bioavailability of phentermine, the preferred length is between a single amino acid and three amino acids.

It should be recognized that the orientation for each of the recited embodiments may be either C-terminus, N-terminus, or side-chained, where the amino acid provides for side chain attachment. It should be understood however, that the bound form is directed to covalent bonding and that salt forms are meant to be included. Additionally, these compounds may be in their salt forms for ease of storage or use in formulations.

The invention provides a method for delivering phentermine to a patient, the patient being a human or a non-human animal, comprising administering to the patient compositions of the invention.

The methods, compounds and compositions of the invention provide many important advantages and advances. The methods and compositions of the invention prevent and/or avoid overdosing (e.g., “spiking”). By assuring dosage reproducibility and/or reducing dosage availability, the invention provides the added advantage of improving patient compliance. The invention provides time-release properties to phentermine. Providing time-release properties also assures dosage reproducibility and/or reduces the number of dosages required.

In a preferred embodiment, the time-release properties provided by the invention are not dependent upon other commonly used delay release or time-release formulations, such as a microencapsulating matrix during manufacturing. This provides a further advantage of reliable dosing and batch-to-batch reproducibility. This embodiment provides a further advantage of time-release properties without heightened dependence on water solubility of the phentermine. As such, the time-release properties do not require further formulations such as the dissolution process involved in an enterically coated active agent controlled by pH.

Another advantage provided by preferred embodiments of the invention is the control of phentermine delivery system with regard to molecular weight, molecular size, particle size or combinations thereof. The control of these physical characteristics provided by this embodiment enables predictable diffusion rates and pharmacokinetics.

In a preferred embodiment of the invention, one or more phentermine-prodrugs are delivered synergistically. In another embodiment, the compositions of the invention protect the phentermine during storage and/or in passage through the stomach. In a more preferred embodiment, the invention provides methods for protecting, controlling delivery, or controlling release of phentermine compounds, or combinations thereof.

In a preferred embodiment, the phentermine conjugates are used in combination with a non-bound phentermine. These combinations may be administered to a patient to treat obesity.

The invention provides the amount of biologically available phentermine in a regulated manner and therefore, side effects known from taking too high a dose of phentermine can be prevented. The amount of free phentermine is regulated by the mechanism that cleaves the amide bond and releases the active drug, thereby minimizing the potential for adverse side effects from high doses. In addition, the absorption of phentermine may be improved.

The invention provides several benefits for phentermine administration, such as but not limited to longer shelf life and the prevention of digestion in the stomach; prolonged pharmacologic effect through delayed release of phentermine; phentermine can be combined together or with adjuvants to produce synergistic effects; enhanced absorption of the phentermine in the intestinal tract; and formulation for digestion by intestinal enzymes, intracellular enzymes or blood serum enzymes.

The carrier peptide can be prepared using conventional techniques. If a specific sequence is desired, an automated peptide synthesizer can be used.

Compositions of the invention may comprise the formation of amides from acids and amines and can be prepared by the examples herein. Throughout the application the figures are meant to describe the general scheme of attaching active agents through different functional groups to a variety of peptide conjugates resulting in different embodiments of the invention. One skilled in the art would recognize other reagents, conditions, and properties necessary to conjugate other active agents to other polypeptides from the schemes that are meant to be non-limiting examples. The figures further represent the different embodiments of the invention with regard to length of the active agent conjugate.

The invention teaches broadly phentermine-prodrugs in combination with unbound phentermine to form compositions and methods of inventions e.g., phentermine-prodrugs and unbound phentermine, etc.

These products will be used at levels similar to those used in treating obesity patients with current treatments. Determining the precise levels to be used in a particular patient may be accomplished using methods well known to those of skill in the art. The compositions will be particularly useful in providing oral dosage formulations. While oral dosage formulations are the preferred embodiment for delivery, methods of delivering known phentermine compounds may also be utilized.

Phentermine may be attached to the carrier peptide through the C-terminus, N-terminus, or side chain of the carrier peptide. Preferably, phentermine is attached to the C-terminus of the carrier peptide. It is preferred that aside from attachment of the carrier peptide to the phentermine neither is further substituted or protected. In one embodiment, the chemical moiety has one or more free carboxy and/or amine terminal and/or side chain group other than the point of attachment to the phentermine. The chemical moiety can be in such a free state, or an ester or salt thereof.

Another embodiment of the invention is a composition or method for safely delivering phentermine comprising providing a therapeutically effective amount of phentermine which has been covalently bound to a chemical moiety wherein said chemical moiety alters the rate of absorption of the phentermine as compared to delivering the unbound phentermine. Another embodiment may also provide a means for reducing drug toxicity by altering the rate of clearance of phentermine.

Another embodiment of the invention is a composition or method for a sustained-release phentermine composition comprising providing phentermine which has been covalently bound to a chemical moiety, wherein said chemical moiety provides release of phentermine at a rate where the level of phentermine is within the therapeutic range but below toxic levels over an extended periods of time, e.g., 8-24 hours or greater.

Another embodiment of the invention is a composition or method for reducing bioavailability or preventing a toxic release profile of phentermine comprising phentermine covalently bound to a chemical moiety wherein said bound phentermine maintains a steady-state serum release curve which provides a therapeutically effective bioavailability but prevents spiking or increase blood serum concentrations compared to unbound phentermine.

Another embodiment of the invention is a composition or method for preventing a Cₘₐₓ spike and/or providing a more consistent release curve for phentermine while still providing a therapeutically effective bioavailability curve comprising phentermine that has been covalently bound to a chemical moiety.

Another embodiment of the invention is a method for reducing or preventing toxicity and/or improving the release and/or providing a steady state of release of a pharmaceutical composition, comprising providing, administering, or prescribing said composition to a human in need thereof, wherein said composition comprises a chemical moiety covalently attached to phentermine.

For each of the recited methods of the invention the following properties may be achieved through bonding phentermine to the chemical moiety. In one embodiment, the toxicity of the compound may be substantially lower than that of the phentermine when delivered in its unbound state or as a salt thereof. In another embodiment, the possibility of overdose/toxicity by oral administration is reduced or eliminated.

The compositions and methods of the invention provide phentermine, which when bound to the chemical moiety provide safer and/or more effective dosages for phentermine through improved bioavailability curves and/or safer Cₘₐₓ and/or reduce area under the curve for bioavailability.

Preferably, the phentermine prodrug exhibits an oral bioavailability of phentermine of at least about 60% AUC (area under the curve), more preferably at least about 70%, 80%, 90%, 95%, 96%, 97%, 98%, 99%, compared to unbound phentermine.

In one embodiment, the phentermine prodrug provides pharmacological parameters (AUC, Cₘₐₓ, Tₘₐₓ, Cₘᵢₙ, and/or t_{1/2}) within 80% to 125%, 80% to 120%, 85% to 125%, 90% to 110%, or increments therein of unbound phentermine or current commercial product utilized for treatment, e.g., Adipex®, Ionamin®, or Duromine®. It should be recognized that the ranges can, but need not be symmetrical, e.g., 85% to 105%.

In another embodiment, the toxicity of the phentermine prodrug is substantially lower than that of the unbound phentermine. For example, in a preferred embodiment, the acute toxicity is 1-fold, 2-fold, 3-fold, 4-fold, 5-fold, 6-fold, 7-fold, 8-fold, 9-fold, 10-fold less, or increments therein less lethal than oral administration of unbound phentermine.

In accordance with the invention and as used herein, the following terms are defined with the following meanings, unless explicitly stated otherwise.

The compounds, compositions and methods of the invention utilize “phentermine conjugates,” which are also referred to as phentermine prodrugs.

Throughout this application the use of “chemical moiety”—sometimes referred to as the “conjugate” or the “carrier”—is meant to include any chemical substance, naturally occurring or synthetic that decreases the pharmacological activity until the phentermine is released including at least carrier peptides, glycopeptides, carbohydrates, lipids, nucleic acids, nucleosides, or vitamins. Preferably, the chemical moiety is generally recognized as safe (“GRAS”).

Throughout this application the use of “carrier peptide” is meant to include naturally occurring amino acids, synthetic amino acids, and combinations thereof. In particular, carrier peptide is meant to include at least a single amino acid, a dipeptide, a tripeptide, a tetrapeptide, an oligopeptide, a polypeptide, or the nucleic acid-amino acids peptides. The carrier peptide can comprise a homopolymer or heteropolymer of naturally occurring or synthetic amino acids.

The use of the term “straight carrier peptide” is meant to include amino acids that are linked via a —C(O)—NH— linkage, also referred to herein as a “peptide bond,” but may be substituted along the side chains of the carrier peptide. Amino acids that are not joined together via a peptide bond or are not exclusively joined through peptide bonds are not meant to fall within the definition of straight carrier peptide.

The use of the term “unsubstituted carrier peptide” is meant to include amino acids that are linked via a —C(O)—NH— linkage, and are not otherwise substituted along the side chains of the carrier peptide. Amino acids that are not joined together via a peptide bond or are not exclusively joined through peptide bonds are not meant to fall within the definition of unsubstituted carrier peptide.

“Oligopeptide” is meant to include from 2 amino acids to 10 amino acids. “Polypeptides” are meant to include from 2 to 50 amino acids.

“Carbohydrates” includes sugars, starches, cellulose, and related compounds. e.g., (CH₂O)ₙ, wherein n is an integer larger than 2 or Cₙ(H₂O)ₙ₋₁, with n larger than 5. More specific examples include for instance, fructose, glucose, lactose, maltose, sucrose, glyceraldehyde, dihydroxyacetone, erythrose, ribose, ribulose, xylulose, galactose, mannose, sedoheptulose, neuraminic acid, dextrin, and glycogen.

A “glycoprotein” is a compound containing carbohydrate (or glycan) covalently linked to protein. The carbohydrate may be in the form of a monosaccharide, disaccharide(s), oligosaccharide(s), polysaccharide(s), or their derivatives (e.g. sulfo- or phospho-substituted).

A “glycopeptide” is a compound consisting of carbohydrate linked to an oligopeptide composed of L- and/or D-amino acids. A glyco-amino-acid is a saccharide attached to a single amino acid by any kind of covalent bond. A glycosyl-amino-acid is a compound consisting of saccharide linked through a glycosyl linkage (O—, N— or S—) to an amino acid.

The “carrier range” or “carrier size” is determined based on the effect desired. It is preferably between one to 12 chemical moieties with one to 8 moieties being preferred. In another embodiment the number of chemical moieties attached is a specific number e.g., 1, 2, 3, 4, 5, 6, 7, 8, 9, or 10, etc. Alternatively, the chemical moiety may be described based on its molecular weight. It is preferred that the conjugate weight is below about 2,500 kD, more preferably below about 1,000 kD and most preferably below about 500 kD.

A “composition” as used herein, refers broadly to any composition containing an phentermine conjugate. A “pharmaceutical composition” refers to any composition containing an phentermine conjugate that only comprises components that are acceptable for pharmaceutical uses, e.g., excludes phentermine conjugates for immunological purposes.

Use of phrases such as “decreased”, “reduced”, “diminished”, or “lowered” includes at least a 10% change in pharmacological activity with respect to at least one ADME characteristic or at least one of AUC, Cₘₐₓ, Tₘₐₓ, Cₘᵢₙ, and t_{1/2}. For instance, the change may also be greater than 25%, 35%, 45%, 55%, 65%, 75%, 85%, 95%, 96%, 97%, 98%, 99%, or other increments.

Use of the phrase “similar pharmacological activity” means that two compounds exhibit curves that have substantially the same AUC, Cₘₐₓ, Tₘₐₓ, Cₘᵢₙ, and/or t_{1/2} parameters, preferably within about 30% of each other, more preferably within about 25%, 20%, 10%, 5%, 2%, 1%, or other increments.

“Cₘₐₓ” is defined as the maximum concentration of free phentermine in the body obtained during the dosing interval.

“Tₘₐₓ” is defined as the time to maximum concentration.

“Cₘᵢₙ” is defined as the minimum concentration of phentermine in the body after dosing.

“t_{1/2}” is defined as the time required for the amount of phentermine in the body to be reduced to one half of its value.

Throughout this application, the term “increment” is used to define a numerical value in varying degrees of precision, e.g., to the nearest 10, 1, 0.1, 0.01, etc. The increment can be rounded to any measurable degree of precision. For example, the range 1 to 100 or increments therein includes ranges such as 20 to 80, 5 to 50, 0.4 to 98, and 0.04 to 98.05.

“Obesity” as used herein, refers broadly to a condition in which ideal weight is exceeded by 20%. Alternatively, obesity in humans includes men with more than 25% body fat and women with more than 30% body fat. Measurement of the percentage of body fat may be made through the body mass index (BMI), which is the weight of the subject in kilograms divided by the height of the subject in meters squared (BMI=kg/m²). A subject with a BMI of 30 or more is obese. Other methods available to measure body fat are known to one of ordinary skill in the art and include, for example, underwater measurement of weight or Dual Energy X-ray Absorptiometry (DEXA).

“Patient” as used herein, refers broadly to any animal that is in need of treatment, most preferably and animal that is obese. The patient may be a clinical patient such as a human or a veterinary patient such as a companion, domesticated, livestock, exotic, or zoo animal. Animals may be mammals, reptiles, birds, amphibians, or invertebrates.

“Mammal” as used herein, refers broadly to any and all warm-blooded vertebrate animals of the class Mammalia, including humans, non-human primates, felines, canines, pigs, horses, sheep, etc.

“Pretreatment” as used herein, refers broadly to any and all preparation, treatment, or protocol that takes place before receiving a phentermine compound or composition of the invention.

“Treating” or “treatment” as used herein, refers broadly to preventing the disease, i.e., causing the clinical symptoms of the disease not to develop in a patient that may be exposed to or predisposed to the disease but does not yet experience or display symptoms of the disease, inhibiting the disease, i.e., arresting or reducing the development of the disease or its clinical symptoms, and/or relieving the disease, i.e., causing regression of the disease or its clinical symptoms. Treatment also encompasses an alleviation of signs and/or symptoms.

“Therapeutically effective amount” as used herein, refers broadly to the amount of a compound that, when administered to a patient for treating obesity is sufficient to effect such treatment for obesity. The “therapeutically effective amount” will vary depending on the compound, the disease and its severity and the age, weight, etc., of the patient to be treated. “Effective dosage” or “Effective amount” of the phentermine compound or composition is that which is necessary to treat or provide prophylaxis for obesity.

“Selection of patients” and “Screening of patients” as used herein, refers broadly to the practice of selecting appropriate patients to receive the treatments described herein. Various factors including but not limited to age, weight, heath history, medications, surgeries, injuries, conditions, illnesses, diseases, infections, gender, ethnicity, genetic markers, polymorphisms, skin color, and sensitivity to phentermine treatment. Still other factors include those used by physicians to determine if a patient is appropriate to receive the treatments described herein.

“Diagnosis” as used herein, refers broadly to the practice of testing, assessing, assaying, and determining whether or not a patient is obese. In particular, one criteria may be the percentage of body weigh due to fat.

Regarding stereochemistry, this patent is meant to cover all compounds discussed regardless of absolute configurations. Thus, natural, L-amino acids are discussed but the use of D-amino acids are also included.

For each of the embodiments recited herein, the carrier peptide may comprise of one or more of the naturally occurring (L-) amino acids: alanine, arginine, asparagine, aspartic acid, cysteine, glycine, glutamic acid, glutamine, histidine, isoleucine, leucine, lysine, methionine, proline, phenylalanine, serine, tryptophan, threonine, tyrosine, and valine. Another preferred amino acid is beta-alanine. In another embodiment the amino acid or peptide is comprised of one or more of the D-form of the naturally occurring amino acids. In another embodiment the amino acid or peptide is comprised of one or more unnatural, non-standard or synthetic amino acids such as, aminohexanoic acid, biphenylalanine, cyclohexylalanine, cyclohexylglycine, diethylglycine, dipropylglycine, 2,3-diaminoproprionic acid, homophenylalanine, homoserine, homotyrosine, naphthylalanine, norleucine, ornithine, pheylalanine(4-fluoro), phenylalanine(2,3,4,5,6 pentafluoro), phenylalanine(4-nitro), phenylglycine, pipecolic acid, sarcosine, tetrahydroisoquinoline-3-carboxylic acid, and tert-leucine. In another embodiment the amino acid or peptide comprises of one or more amino acid alcohols. In another embodiment the amino acid or peptide comprises of one or more N-methyl amino acids.

In another embodiment, the specific carriers may have one or more of amino acids substituted with one of the 20 naturally occurring amino acids. It is preferred that the substitution be with an amino acid which is similar in structure or charge compared to the amino acid in the sequence. For instance, isoleucine (Ile)[I] is structurally very similar to leucine (Leu)[L], whereas, tyrosine (Tyr) [Y] is similar to phenylalanine (Phe)[F], whereas serine (Ser)[S] is similar to threonine (Thr)[T], whereas cysteine (Cys)[C] is similar to methionine (Met)[M], whereas alanine (Ala)[A] is similar to valine (Val)[V], whereas lysine (Lys)[K] is similar to arginine (Arg)[R], whereas asparagine (Asn)[N] is similar to glutamine (Gln)[Q], whereas aspartic acid (Asp)[D] is similar to glutamic acid (Glu)[E], whereas histidine (His)[H] is similar to proline (Pro)[P], and glycine (Gly)[G] is similar to tryptophan (Trp)[W]. In the alternative the preferred amino acid substitutions may be selected according to hydrophilic properties (i.e., polarity) or other common characteristics associated with the 20 essential amino acids. While preferred embodiments utilize the 20 natural amino acids for their GRAS characteristics, it is recognized that minor substitutions along the amino acid chain that do not affect the essential characteristics of the amino are also contemplated.

Herein is a list or where amino acids are grouped according to the characteristics of the side chains:

Aliphatic: Alanine, Glycine, Isoleucine, Leucine, Proline, Valine

Aromatic: Phenylalanine, Tryptophan, Tyrosine

Acidic: Aspartic acid, Glutamic acid

Basic: Arginine, Histidine, Lysine

Hydroxylic: Serine, Threonine

Sulphur-containing: Cysteine, Methionine

Amidic (containing amide group): Asparagine, Glutamine.

The phentermine conjugate may also be in salt form. Pharmaceutically acceptable salts, e.g., non-toxic, inorganic and organic acid addition salts, are known in the art. Exemplary salts include, but are not limited to, 2-hydroxyethanesulfonate, 2-naphthalenesulfonate, 3-hydroxy-2-naphthoate, 3-phenylpropionate, acetate, adipate, alginate, amsonate, aspartate, benzenesulfonate, benzoate, besylate, bicarbonate, bisulfate, bitartrate, borate, butyrate, calcium edetate, camphorate, camphorsulfonate, camsylate, carbonate, citrate, clavulariate, cyclopentanepropionate, digluconate, dodecylsulfate, edetate, edisylate, estolate, esylate, ethanesulfonate, finnarate, gluceptate, glucoheptanoate, gluconate, glutamate, glycerophosphate, glycollylarsanilate, hemisulfate, heptanoate, hexafluorophosphate, hexanoate, hexylresorcinate, hydrabamine, hydrobromide, hydrochloride, hydroiodide, hydroxynaphthoate, isothionate, lactate, lactobionate, laurate, laurylsulphonate, malate, maleate, mandelate, mesylate, methanesulfonate, methylsulfate, mucate, naphthylate, napsylate, nicotinate, nitrate, N-methylglucamine ammonium salt, oleate, oxalate, palmitate, pamoate, pantothenate, pectinate, phosphate, phosphateldiphosphate, picrate, pivalate, polygalacturonate, propionate, p-toluenesulfonate, saccharate, salicylate, stearate, subacetate, succinate, sulfate, sulfosaliculate, suramate, tannate, tartrate, teoclate, thiocyanate, tosylate, triethiodide, undecanoate, and valerate salts, and the like.

In the invention, phentermine may be covalently attached to the peptide via a ketone group and a linker. This linker may be a small linear or cyclic molecule containing 2-6 atoms with one or more heteroatoms (such as O, S, N) and one or more functional groups (such as amines, amides, alcohols or acids) or may be made up of a short chain of either amino acids or carbohydrates). For example, glucose would be suitable as a linker.

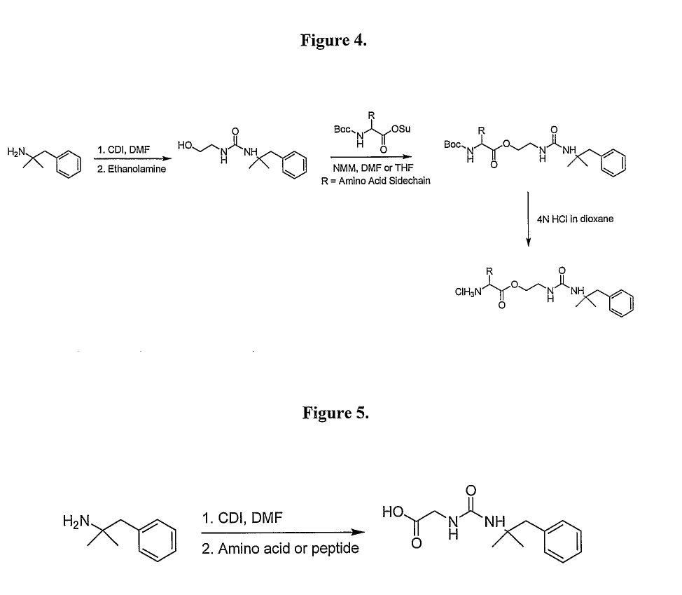

In yet another embodiment of the invention, linkers can be selected from the group of all chemical classes of compounds such that virtually any side chain of the peptide can be attached. The linker should have a functional pendant group, such as a carboxylate, an alcohol, thiol, oxime, hydraxone, hydrazide, or an amine group, to covalently attach to the carrier peptide. Examples of linking organic compounds to the N-terminus type of a peptide include, but are not limited to, the attachment of naphthylacetic acid to LH-RH, coumarinic acid to opioid peptides and 1,3-dialkyl-3-acyltriazenes to tetragastrin and pentagastrin. As another example, there are known techniques for forming peptide linked biotin and peptide linked acridine. FIG. 4 illustrates an embodiment where phentermine is covalently attached to a carrier peptide through a linker.

In addition to the phentermine prodrug, the pharmaceutical compositions of the invention may further comprise one or more pharmaceutical additives. Pharmaceutical additives include a wide range of materials including, but not limited to diluents and bulking substances, binders and adhesives, lubricants, glidants, plasticizers, disintegrants, carrier solvents, buffers, colorants, flavorings, sweeteners, preservatives and stabilizers, adsorbents, and other pharmaceutical additives known in the art.

Lubricants include, but are not limited to, magnesium stearate, calcium stearate, zinc stearate, powdered stearic acid, glyceryl monostearate, glyceryl palmitostearate, glyceryl behenate, silica, magnesium silicate, colloidal silicon dioxide, titanium dioxide, sodium benzoate, sodium lauryl sulfate, sodium stearyl fumarate, hydrogenated vegetable oil, talc, polyethylene glycol, and mineral oil.

Surface agents for formulation include, but are not limited to, sodium lauryl sulfate, dioctyl sodium sulfosuccinate, triethanolamine, polyoxyethylene sorbitan, poloxalkol, and quarternary ammonium salts; excipients such as lactose, mannitol, glucose, fructose, xylose, galactose, sucrose, maltose, xylitol, sorbitol, chloride, sulfate and phosphate salts of potassium, sodium, and magnesium; gelling agents such as colloidal clays; thickening agents such as gum tragacanth or sodium alginate, effervescing mixtures; and wetting agents such as lecithin, polysorbates or laurylsulphates.

Colorants can be used to improve appearance or to help identify the pharmaceutical composition. See 21 C.F.R., Part 74. Exemplary colorants include D&C Red No. 28, D&C Yellow No. 10, FD&C Blue No. 1, FD&C Red No. 40, FD&C Green #3, FD&C Yellow No. 6, and edible inks.

In embodiments where the pharmaceutical composition is compacted into a solid dosage form, e.g., a tablet, a binder can help the ingredients hold together. Binders include, but are not limited to, sugars such as sucrose, lactose, and glucose; corn syrup; soy polysaccharide, gelatin; povidone (e.g., Kollidon®, Plasdone®); Pullulan; cellulose derivatives such as microcrystalline cellulose, hydroxypropylmethyl cellulose (e.g., Methocel®), hydroxypropyl cellulose (e.g., Klucel®), ethylcellulose, hydroxyethyl cellulose, carboxymethylcellulose sodium, and methylcellulose; acrylic and methacrylic acid co-polymers; carbomer (e.g., Carbopol®); polyvinylpolypyrrolidine, polyethylene glycol (Carbowax®); pharmaceutical glaze; alginates such as alginic acid and sodium alginate; gums such as acacia, guar gum, and arabic gums; tragacanth; dextrin and maltodextrin; milk derivatives such as whey; starches such as pregelatinized starch and starch paste; hydrogenated vegetable oil; and magnesium aluminum silicate, as well as other conventional binders known to persons skilled in the art. Exemplary non-limiting bulking substances include sugar, lactose, gelatin, starch, and silicon dioxide.

Glidants can improve the flowability of non-compacted solid dosage forms and can improve the accuracy of dosing. Glidants include, but are not limited to, colloidal silicon dioxide, fumed silicon dioxide, silica gel, talc, magnesium trisilicate, magnesium or calcium stearate, powdered cellulose, starch, and tribasic calcium phosphate.

Plasticizers include, but are not limited to, hydrophobic and/or hydrophilic plasticizers such as, diethyl phthalate, butyl phthalate, diethyl sebacate, dibutyl sebacate, triethyl citrate, acetyltriethyl citrate, acetyltributyl citrate, cronotic acid, propylene glycol, castor oil, triacetin, polyethylene glycol, propylene glycol, glycerin, and sorbitol. Plasticizers are particularly useful for pharmaceutical compositions containing a polymer and in soft capsules and film-coated tablets.

Flavorings improve palatability and may be particularly useful for chewable tablet or liquid dosage forms. Flavorings include, but are not limited to maltol, vanillin, ethyl vanillin, menthol, citric acid, fumaric acid, ethyl maltol, and tartaric acid. Sweeteners include, but are not limited to, sorbitol, saccharin, sodium saccharin, sucrose, aspartame, fructose, mannitol, and invert sugar.

Preservatives and/or stabilizers improving storagability include, but are not limited to, alcohol, sodium benzoate, butylated hydroxy toluene, butylated hydroxyanisole, and ethylenediamine tetraacetic acid.

Disintegrants can increase the dissolution rate of a pharmaceutical composition. Disintegrants include, but are not limited to, alginates such as alginic acid and sodium alginate, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), polyvinylpolypyrrolidine (Plasone-XL®), guar gum, magnesium aluminum silicate, methyl cellulose, microcrystalline cellulose, polacrilin potassium, powdered cellulose, starch, pregelatinized starch, sodium starch glycolate (e.g., Explotab®, Primogel®).

Diluents increase the bulk of a dosage form and may make the dosage form easier to handle. Exemplary diluents include, but are not limited to, lactose, dextrose, saccharose, cellulose, starch, and calcium phosphate for solid dosage forms, e.g., tablets and capsules; olive oil and ethyl oleate for soft capsules; water and vegetable oil for liquid dosage forms, e.g., suspensions and emulsions. Additional suitable diluents include, but are not limited to, sucrose, dextrates, dextrin, maltodextrin, microcrystalline cellulose (e.g., Avicel®), microfine cellulose, powdered cellulose, pregelatinized starch (e.g., Starch 1500®), calcium phosphate dihydrate, soy polysaccharide (e.g., Emcosoy®), gelatin, silicon dioxide, calcium sulfate, calcium carbonate, magnesium carbonate, magnesium oxide, sorbitol, mannitol, kaolin, polymethacrylates (e.g., Eudragit®), potassium chloride, sodium chloride, and talc.

In embodiments where the pharmaceutical composition is formulated for a liquid dosage form, the pharmaceutical composition may include one or more solvents. Suitable solvents include, but are not limited to, water; alcohols such as ethanol and isopropyl alcohol; methylene chloride; vegetable oil; polyethylene glycol; propylene glycol; and glycerin or mixing and combination thereof.

The pharmaceutical composition can comprise a buffer. Buffers include, but are not limited to, lactic acid, citric acid, acetic acid, sodium lactate, sodium citrate, and sodium acetate.

Hydrophilic polymers suitable for use in the sustained release formulation include: one or more natural or partially or totally synthetic hydrophilic gums such as acacia, gum tragacanth, locust bean gum, guar gum, or karaya gum, modified cellulosic substances such as methylcellulose, hydroxomethylcellulose, hydroxypropyl methylcellulose, hydroxypropyl cellulose, hydroxyethylcellulose, carboxymethylcellulose; proteinaceous substances such as agar, pectin, carrageen, and alginates; and other hydrophilic polymers such as carboxypolymethylene, gelatin, casein, zein, bentonite, magnesium aluminum silicate, polysaccharides, modified starch derivatives, and other hydrophilic polymers known to those of skill in the art or a combination of such polymers.

One of ordinary skill in the art would recognize a variety of structures such as bead constructions and coatings, useful for achieving particular release profiles. It is also possible for the dosage form to combine any forms of release known to persons of ordinary skill in the art. These include immediate release, extended release, pulse release, variable release, controlled release, timed release, sustained release, delayed release, long acting, and combinations thereof. The ability to obtain immediate release, extended release, pulse release, variable release, controlled release, timed release, sustained release, delayed release, long acting characteristics, and combinations thereof is known in the art, See, e.g., U.S. Pat. No. 6,913,768.

However, it should be noted that the phentermine conjugate controls the release of phentermine into the digestive tract over an extended period of time resulting in an improved profile when compared to immediate release combinations and reduces and/or prevents toxicity without the addition of the above additives. In a preferred embodiment no further sustained release additives are required to achieve a blunted or reduced pharmacokinetic curve while achieving therapeutically effective amounts of phentermine release.

The dose range for adult human beings will depend on a number of factors including the age, weight and condition of the patient and the administration route. Tablets and other forms of presentation provided in discrete units conveniently contain a daily dose, or an appropriate fraction thereof, of the phentermine conjugate. The dosage form can contain a dose of about 2.5 mg to about 500 mg, about 10 mg to about 300 mg, about 10 mg to about 100 mg, about 25 mg to about 75 mg, or increments therein. In a preferred embodiment, the dosage form contains 5 mg, 10 mg, 25 mg, 30 mg, 37.5 mg, 50 mg, or 100 mg of a phentermine prodrug.

Tablets and other dosage forms provided in discrete units can contain a daily dose, or an appropriate fraction thereof, of one or more phentermine prodrugs.

Compositions of the invention may be administered in a partial, i.e., fractional dose, one or more times during a 24 hour period, a single dose during a 24 hour period of time, a double dose during a 24 hour period of time, or more than a double dose during a 24 hour period of time. Fractional, double or other multiple doses may be taken simultaneously or at different times during the 24-hour period. The doses may be uneven doses with regard to one another or with regard to the individual components at different administration times. Preferably, a single dose is administered once daily.

Likewise, the compositions of the invention may be provided in a blister pack or other such pharmaceutical package. Further, the compositions of the present inventive subject matter may further include or be accompanied by indicia allowing individuals to identify the compositions as products for a prescribed treatment. The indicia may further additionally include an indication of the above specified time periods for administering the compositions. For example the indicia may be time indicia indicating a specific or general time of day for administration of the composition, or the indicia may be a day indicia indicating a day of the week for administration of the composition. The blister pack or other combination package may also include a second pharmaceutical product.

The compounds of the invention can be administered by a variety of dosage forms. Any biologically acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspension in an aqueous liquid or a non-aqueous liquid, emulsions, tablets, syringes, multi-layer tablets, bi-layer tablets, capsules, soft gelatin capsules, hard gelatin capsules, caplets, lozenges, chewable lozenges, beads, powders, granules, particles, microparticles, dispersible granules, cachets, suppositories, creams, topicals, inhalants, aerosol inhalants, patches, particle inhalants, implants, depot implants, ingestibles, injectables (including subcutaneous, intramuscular, intravenous, and intradermal), infusions, emulsions, health bars, confections, animal feeds, cereals, yoghurts, cereal coatings, foods, nutritive foods, functional foods and combinations thereof. Preferably, said composition may be in the form of any of the known varieties of tablets (e.g., chewable tablets, conventional tablets, film-coated tablets, compressed tablets), capsules, liquid dispersions for oral administration (e.g., syrups, emulsions, solutions or suspensions).

However, the most effective means for delivering the phentermine compounds of the invention is orally, to permit maximum release of phentermine to provide therapeutic effectiveness and/or sustained release. When delivered by the oral route phentermine is released into circulation, preferably over an extended period of time as compared to phentermine alone.

It is preferred that the phentermine conjugate be compact enough to allow for a reduction in overall administration size. The smaller size of the phentermine prodrug dosage forms promotes ease of swallowing.

For oral administration, fine powders or granules containing diluting, dispersing and/or surface-active agents may be presented in a draught, in water or a syrup, in capsules or sachets in the dry state, in a non-aqueous suspension wherein suspending agents may be included, or in a suspension in water or a syrup. Where desirable or necessary, flavoring, preserving, suspending, thickening or emulsifying agents can be included.

Preferably, the composition of the invention is in a form suitable for oral administration. Commonly applied oral formulations are further described in US2003/0050344 that is hereby incorporated by reference in its entirety. Additional oral formulations are described in the U.S. Pharmacopeia, Vol. 28, 2005 and can be found at http://www.fda.gov/cder/dsm/DRG/drg00201.1htm.

Accordingly, the invention also provides methods comprising providing, administering, prescribing, or consuming a phentermine prodrug. The invention also provides pharmaceutical compositions comprising a phentermine prodrug. The formulation of such a pharmaceutical composition can optionally enhance or achieve the desired release profile.

It will be appreciated that the pharmacological activity of the compositions of the invention can be demonstrated using standard pharmacological models that are known in the art. For each of the described embodiments one or more characteristics as described throughout the specification may be realized. It should also be recognized that the compounds and compositions described throughout the specification may be utilized for a variety of novel methods of treatment, reduction of toxicity, improved release profiles, etc. An embodiment may obtain, one or more of: a conjugate with toxicity of phentermine that is substantially lower than that of unbound phentermine.

### EXAMPLES

Any feature of the above-describe embodiments can be used in combination with any other feature of the above-described embodiments. Synthesis of amino acid and peptide conjugates may be verified using the following analytical methods: Nuclear Magnetic Resonance, High Resolution Mass Spectroscopy or Elemental Analysis and melting point or differential scanning calorimetry (DSC).

In order to facilitate a more complete understanding of the invention, Examples are provided below. However, the scope of the invention is not limited to specific embodiments disclosed in these Examples, which are for purposes of illustration only.

### Example 1

### General Synthetic Pathway of Phentermine to Amino Acids and Peptides

Schemes of synthesis are also described in FIGS. 1-5.

To a mixture of any N-Boc or acid labile protected amino acid or peptide and phentermine would be added a co-base (4-methylmorpholine) and an appropriate solvating agent. This reaction mixture would then be stirred until reaction was complete. Reaction would then be quenched with water and excess solvent removed. Crude material would be extracted into a non-polar organic solvent or purified directly using reverse phase HPLC.

To the isolated protected intermediate would be added the appropriate deprotecting acid (4N HCl in dioxane, TFA). Reaction would be monitored for completion and the corresponding salt would be isolated by solvent removal.

1. A composition comprising phentermine and a peptide carrier comprising one to ten amino acids, wherein phentermine is conjugated to either the C-terminus of the peptide carrier or to an amino acid side chain in the peptide carrier.

2. (canceled)

3. The composition of claim 1, wherein said peptide carrier is Ala, Gly, Leu, Val, He, Phe, Pro, Lys, Asp, Glu, Ser, Thr, Tyr, Ala-Ala, Gly-Gly, Phe-Phe, Glu-Glu, Tyr-Tyr, Ala-Ala-Ala, or Gly-Gly-Gly.

4. The composition of claim 1, wherein said peptide carrier is a single amino acid.

5. The composition of claim 1, wherein said composition is in salt form.

6. The composition of claim 1, wherein said composition is in ester form.

7. The composition of claim 5, wherein said salt form is a HCl, acetate, sulfate, mesylate, citrate, nitrate, or phosphate salt.

8. The composition of claim 1, further comprising a pharmaceutically acceptable excipient and wherein said composition is in oral dosage form.

9. The composition of claim 8, wherein said oral dosage form is a tablet, a capsule, a caplet, an liquid oral dosage.

10. A method of treating obesity comprising administering the composition of claim 1 to a patient in need thereof.

11. Lys-phentermine.

12. X-Lys-phentermine wherein X is one of the naturally occurring amino acids.

13. The salt or ester of the compound of claim 11 or claim 12.

14. A phentermine prodrug of Formula I:
P—Xₙ-Z₁₁₁ (I) wherein P is an phentermine; each X is independently a chemical moiety; each Z is independently a chemical moiety that acts as an adjuvant and is different from at least one X; n is an increment from 1 to 50, preferably 1 to 10; and m is an increment from 0 to 50, preferably 0.
